(19) United States
(12) Patent Application Publication
(10) Pub. No.: US 2013/0204360 A1
(43) Pub. Date: Aug. 8, 2013

(54) Invertible Tissue Valve And Method

(71) Applicant: HLT, INC., Maple Grove, MN (US)

(72) Inventor: John P. Gainor, Mendota Heights, MN (US)

(73) Assignee: HLT, INC., Maple Grove, MN (US)

(21) Appl. No.: 13/757,683

(22) Filed: Feb. 1, 2013

Related U.S. Application Data
(60) Provisional application No. 61/593,817, filed on Feb. 1, 2012.

(51) Int. Cl. A61F 2/24

(52) U.S. Cl. CPC A61F 2/2403
USPC 623/2.18

(57) ABSTRACT

A prosthetic valve assembly that includes a stent, a tissue sleeve and an anchoring mechanism. By loading the three components of the valve assembly into a delivery catheter in a series formation, such that no two components are located within each other, the size of the delivery catheter can be reduced.

### RELATED APPLICATIONS

This application claims priority to U.S. Provisional Application Ser. No. 61/593,817 filed Feb. 1, 2012 entitled Invertable Tissue Valve, which is hereby incorporated herein by reference in its entirety.

### BACKGROUND OF THE INVENTION

Replacing heart valves with prosthetic valves was, until recently, a complicated surgical procedure that involved cutting open the chest, establishing blood flow through a blood pump, stopping the heart, etc. This complicated procedure, even when performed perfectly, required extensive recovery time due to the invasiveness and damage done to access the implantation site. Additionally, the risk of infection or other complications is extremely high.

Numerous advancements have been made to develop prosthetic valves that can be implanted percutaneously, using a catheter to snake the prosthetic valve through the vasculature to the implantation site. If successful, the recovery time is greatly minimized relative to conventional open-heart surgery.

A designer of a percutaneously-delivered prosthetic valve is faced with numerous challenges, however. First and foremost is designing a prosthetic valve that can be compressed enough to be inserted into a catheter small enough to be navigated to the valve site through the vasculature. Other challenges include anchoring the valve at the valve site so the valve does not migrate after release; including a support structure for the valve that is robust enough to push the native, often calcified valve out of the way and prevent it from later interfering with the function of the new valve; ensuring that the new valve allows proper flow in a desired direction and effectively stops flow in the opposite direction; ensuring that no blood flows around the sides of the implanted device (this is known as perivalvular leakage); designing a prosthetic valve device that does not fail due to fatigue after hundreds of thousands of cycles of leaflet function; designing a valve that meets all of these criteria and can still be manufactured economically; and the list goes on.

These prosthetic valves, and their respective delivery catheters, are designed to replace a particular native valve. One native valve that presents unique challenges is the aortic valve. The aortic valve controls blood flow from the left ventricle into the aorta. Reaching the aortic valve percutaneously is typically accomplished using one of two approaches.

The first approach is a transfemoral retrograde approach whereby the femoral artery is accessed near the groin of the patient, and followed upstream to the aortic valve. The transfemoral approach is retrograde because travel is against the flow of blood and thus the downstream side of the aortic valve is reached first.

The second approach is a transapical antegrade approach which can be performed via a left anterolateral minithoracotomy. This approach punctures the apex of the heart to provide direct access to the left ventricle. Thus, the aortic valve is approached from the upstream (antegrade) side.

There are advantages and disadvantages to both approaches. The transfemoral approach is considered less invasive because the heart is not punctured. However, the navigation is much longer and access to the aortic valve requires the necessarily longer delivery catheter to follow the curve of the aortic arch at the end of the path to the valve. Thus, the delivery catheter must be more maneuverable and the prosthetic valve must not interfere with the maneuverability of the catheter while the valve is loaded into it. The transapical approach requires puncturing the myocardial sac and the apex of the heart and traversing the chest cavity. In each of these cases, the heart is beating as well, adding more difficulty to the procedure.

### OBJECTS AND SUMMARY OF THE INVENTION

One aspect of the invention is directed to a prosthetic valve device for use in replacing a native aortic valve using a retrograde approach.

Another aspect of the invention is directed to a prosthetic valve device that is sized to replace an aortic valve and capable of being delivered using a small, flexible catheter.

Another aspect of the invention is directed to a prosthetic valve device that comprises two components positioned in series (spaced apart axially) in a delivery catheter to reduce the size of the delivery catheter required.

Another aspect of the invention is directed to a prosthetic valve device that comprises two components positioned in series during navigation whereby the two components can be located together upon delivery to the target site.

One aspect of the invention provides a device for replacing a native valve comprising: a stent; a tissue sleeve; and, an anchoring mechanism usable to secure said tissue sleeve within said stent; wherein, in a configuration inside a delivery catheter, said anchoring mechanism is not located within said stent; and wherein, in a deployed configuration, said tissue sleeve is located within said stent.

Another aspect of the invention provides that said tissue sleeve is inverted (i.e. inside-out) relative to said configuration inside said delivery catheter.

Another aspect of the invention provides that the tissue sleeve is connected to said stent at a first end and connected to said anchoring mechanism at a second end, opposite said first end.

Another aspect of the invention provides an anchoring mechanism that comprises a wireform.

Another aspect of the invention provides an anchoring mechanism that comprises a ring.

Another aspect of the invention provides an anchoring mechanism that comprises a ring and a wireform.

Another aspect of the invention provides an anchoring mechanism that comprises a ring attached to a first end of said tissue sleeve and a second ring attached to an opposite side of said tissue sleeve.

Another aspect of the invention provides a tissue sleeve that comprises valve leaflets.

Another aspect of the invention provides a tissue sleeve that comprises pinch points that result in the formation of valve leaflets when implanted.

Another aspect of the invention provides a method for replacing a native blood valve comprising: expanding a stent within said native valve; anchoring a tissue sleeve at a proximal end of said stent; advancing said tissue sleeve into said stent, resulting in an inversion of said tissue sleeve; and allowing blood to flow through said tissue sleeve in a proximal direction while not allowing blood to flow through said tissue sleeve in a distal direction.

In one aspect of the invention a method is provided wherein expanding a stent within said native valve comprises expanding a stent within said native valve prior to introducing said tissue sleeve at a proximal end of said stent.

In another aspect of the invention a method is provided wherein anchoring a tissue sleeve at a proximal end of said stent occurs prior to the step of expanding a stent within said native valve.

In another aspect of the invention a method is provided wherein anchoring a tissue sleeve at a proximal end of said stent comprises attaching said tissue sleeve to said proximal end of said stent.

In another aspect of the invention a method is provided wherein anchoring a tissue sleeve at a proximal end of said stent comprises allowing a ring to expand at a proximal end of said stent, said ring attached to said tissue sleeve.

In another aspect of the invention a method is provided wherein allowing blood to flow through said tissue sleeve in a proximal direction while not allowing blood to flow through said tissue sleeve in a distal direction comprises providing valve leaflets attached to said tissue sleeve such that said valve leaflets are located on an outside surface of said tissue sleeve prior to said inversion and on an inside of said tissue sleeve after inversion.

In another aspect of the invention a method is provided wherein allowing blood to flow through said tissue sleeve in a proximal direction while not allowing blood to flow through said tissue sleeve in a distal direction comprises expanding a wireform within said tissue sleeve, said wireform creating valve leaflets in said tissue sleeve when expanded.

In another aspect of the invention a method is provided wherein allowing blood to flow through said tissue sleeve in a proximal direction while not allowing blood to flow through said tissue sleeve in a distal direction comprises providing pinch points in a proximal end of said tissue sleeve, after said tissue sleeve is inverted, said pinch point causing blood flow to form valve leaflets out of said tissue sleeve when blood is flowing in a distal direction.

In another aspect of the invention a method is provided further comprising the step of anchoring said tissue sleeve near a distal end of said stent after the step of advancing said tissue sleeve into said stent.

One aspect of the invention provides a valve assembly for implantation within a stent comprising: a tissue sleeve; at least one anchoring mechanism for securing said tissue sleeve within said stent; wherein said tissue sleeve is connected to said at least one anchoring mechanism.

Another aspect of the invention provides a valve assembly wherein said tissue sleeve is attached to a proximal end of said stent.

Another aspect of the invention provides a valve assembly wherein said at least one anchoring mechanism comprises a wireform.

Another aspect of the invention provides a valve assembly wherein said at least one anchoring mechanism comprises at least one expandable ring.

Another aspect of the invention provides a valve assembly wherein said at least one expandable ring comprises a first expandable ring at one end of said tissue sleeve and a second expandable ring at another end of said tissue sleeve.

Another aspect of the invention provides a valve assembly wherein said tissue sleeve comprises valve leaflets.

Another aspect of the invention provides a valve assembly wherein said tissue sleeve forms valve leaflets when said at least one anchoring mechanism is expanded.

Another aspect of the invention provides a valve assembly wherein said tissue sleeve comprises valve leaflets that become function after said tissue sleeve is inverted.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a side view of an embodiment of the invention;

FIGS. 2A-2D illustrate a delivery sequence for the embodiment of FIG. 1;

FIGS. 3A-F illustrate a delivery sequence for an embodiment of the invention;

FIG. 4 is a side view of an embodiment of the invention;

FIG. 5 is a side view of an embodiment of the invention;

FIG. 6 is a side view of an embodiment of the invention;

FIGS. 7A-D illustrate a delivery sequence for an embodiment of the invention;

FIGS. 8A-D illustrate a delivery sequence for an embodiment of the invention; and

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

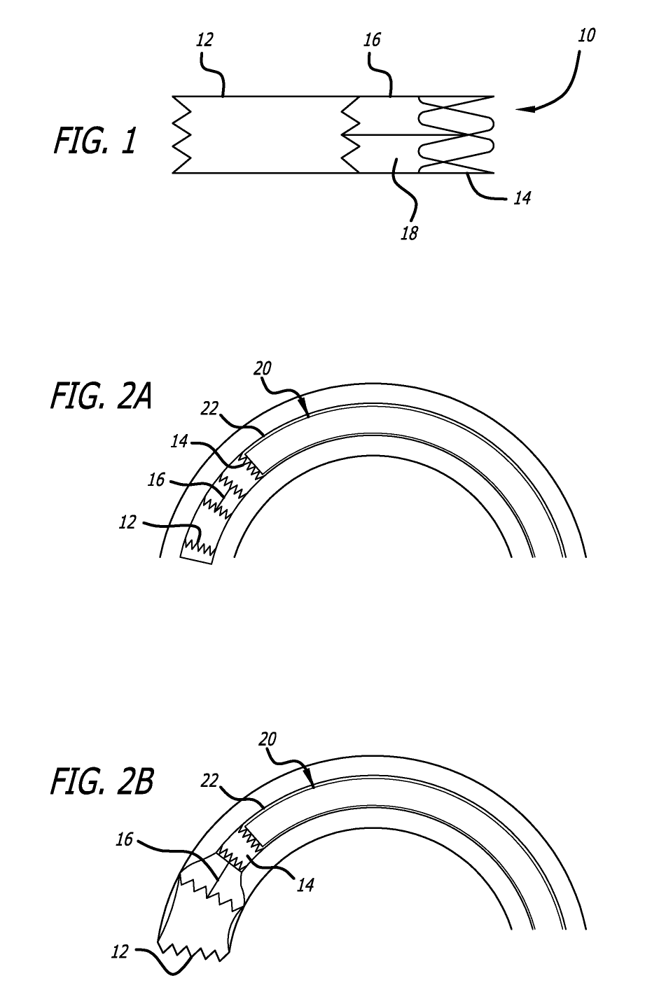

Referring first to FIG. 1 there is shown a device 10 of the invention. Device 10 generally includes a stent 12 connected to a valve assembly that includes a valve frame 14 and tissue connectors 16. Tissue 18 forms a prosthetic valve, shaped by the valve frame 14. The tissue connectors act as a stop when the valve frame 14 is advanced into the stent 12 during delivery. Note that the tissue 18 between the stent 12 and the valve frame 14 prevents blood from flowing around the valve frame 14. Thus perivalvular leakage is avoided.

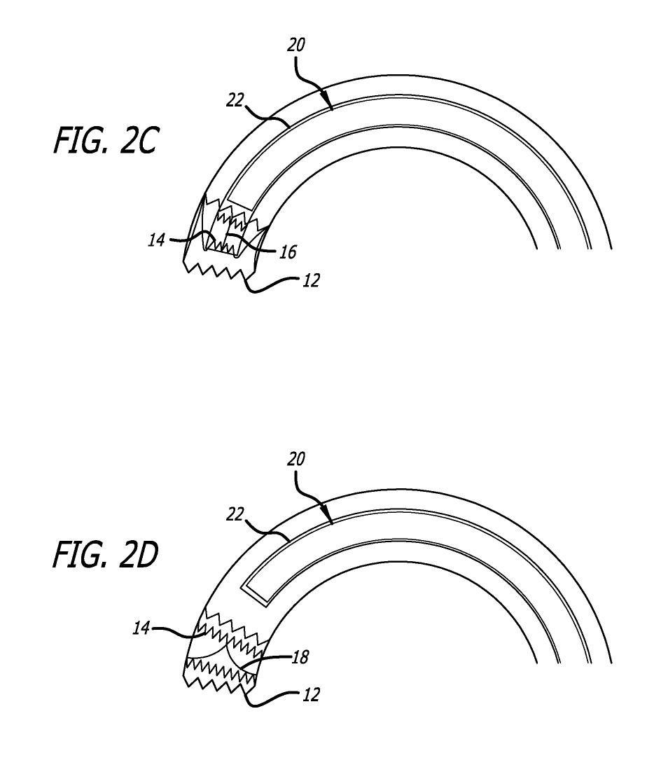

Thus, delivery of the device 10, as illustrated in FIGS. 2A-2D would involve navigation a catheter 20 to the valve site (FIG. 2A), retracting a restraining sheath 22 until the stent 12 is released and allowed to expand (FIG. 2B), advancing the still-constrained valve frame 14 into the expanded stent 12 until the tissue connectors 16 prevent further distal movement (FIG. 2C), retracting the restraining sheath 22 until the valve frame 14 is released, allowing the valve frame 14 to expand within the stent 12 (FIG. 2D).

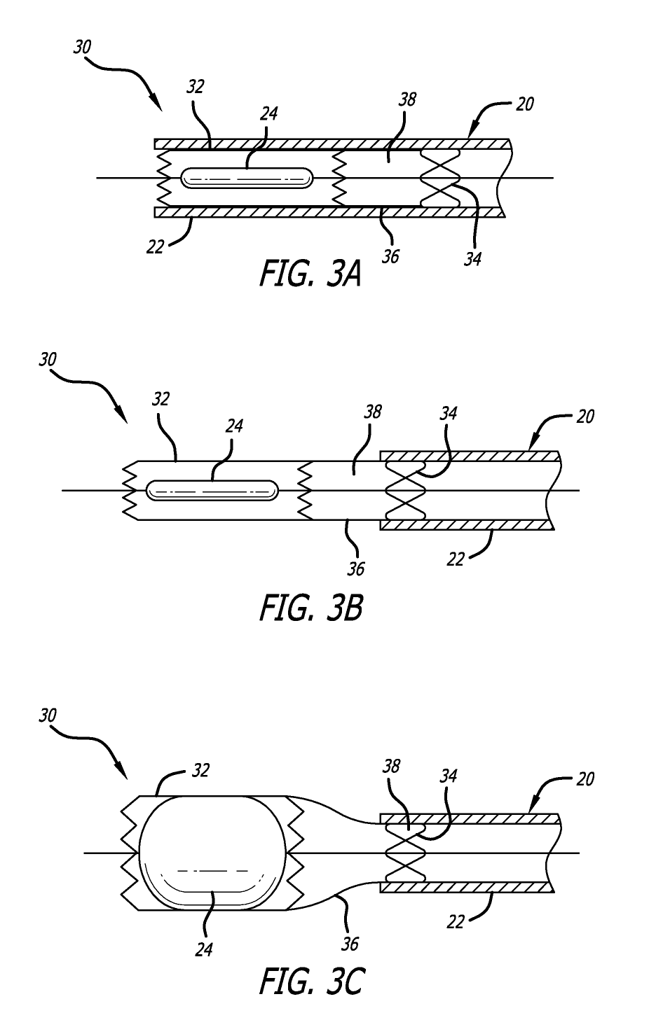

FIGS. 3A-3E illustrate an embodiment 30 of the invention wherein the stent 32 is balloon-expandable. Device 30 generally includes a stent 32 connected to a valve frame 34 with tissue connectors 36. Tissue 38 forms a prosthetic valve, shaped by the valve frame 34. The tissue connectors act as a stop when the valve frame 34 is advanced into the stent 32 during delivery. Note that the tissue 38 between the stent 32 and the valve frame 34 prevents blood from flowing around the valve frame 34. Thus perivalvular leakage is avoided.

Thus, delivery of the device 10, as illustrated in FIGS. 3A-3E would involve navigation a catheter 20 to the valve site (FIG. 3A), retracting a restraining sheath 22 until the stent 32 is released (FIG. 3B), inflating a balloon 24 within the stent 32 to expand the stent 32 (FIG. 3C), deflating the balloon 24 (FIG. 3D), advancing the still-constrained valve frame 34 into the expanded stent 32 until the tissue connectors 36 prevent further distal movement (FIG. 3E), retracting the restraining sheath 22 until the valve frame 14 is released, allowing the valve frame 14 to expand within the stent 12 (FIG. 3F).

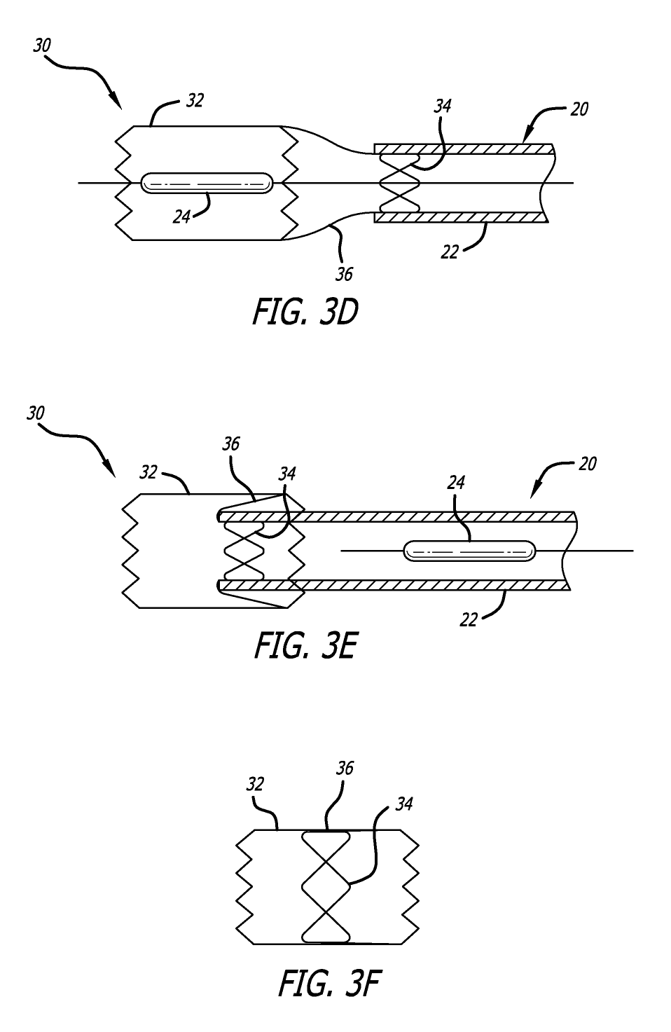

FIG. 4 shows an embodiment 40 of a device of the invention. The device 40 shown in FIG. 4 Device 40 includes a stent 42, and a valve assembly that includes a wireform 44 and tissue 46. Rather than sewing the tissue to the wireform 44, thereby creating a valve frame for the prosthetic valve, the tissue 46 is attached to the wireform 44 at attachment points 50. Valve leaflets 48 are incorporated into the tissue 46 spanning between the stent 42 and the wireform 44. In the delivery configuration shown in FIG. 4, the valve leaflets are located on the outside of the tissue sleeve 46. During delivery, using one of the procedures described above, as the wireform 44 is inserted inside the expanded stent 42, the tissue 46 and the leaflets 48 are inverted (i.e. turned inside-out) so that the valve leaflets are on the inside of the tissue sleeve 46. The wireform 44 is then expanded against the inside of the tissue sleeve 46, and aligned with the valve leaflets 48 so as not to interfere with their function. Thus the tissue sleeve 46, leaflets 48, and wireform 44, together form the prosthetic valve.

Device 40 allows a prosthetic valve to be formed using significantly less tissue material, as there is no need for two layers of tissue around the perimeter of the device after implant. Additionally, device 40 makes it possible to establish flow regulation through the device even at the intermediate stage of device implant.

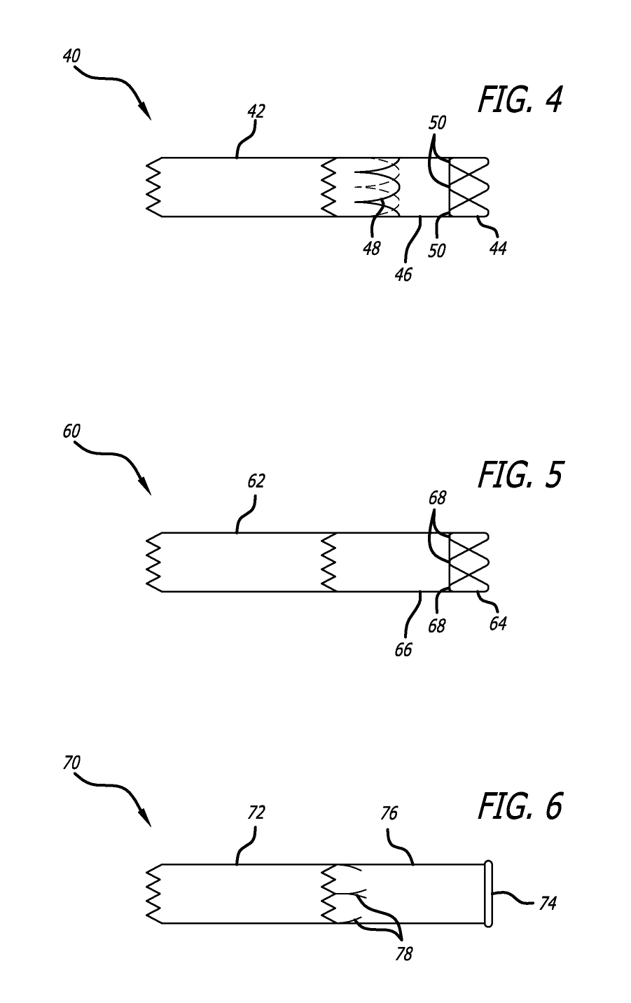

FIG. 5 shows a device 60 that is similar to that of FIG. 4 except that it does not include valve leaflets. The device 60 includes a stent 62, and a valve assembly that includes a wireform 64, and a tissue sleeve 66. Tissue sleeve 66 is simply a tube of tissue. Once the device 60 is delivered, using any of the methods described above, the tissue sleeve 66 is inverted inside the stent 62 and the wireform 64 is expanded. When the wireform 64 is expanded inside the tissue sleeve 66, the wireform 64 creates leaflets due to the shape of the wireform 64. In this way, alignment of the wireform 64 inside the tissue sleeve 66 is not as critical as the embodiment 50 described above. Rather, one would merely need to ensure that there is enough tissue in the tissue sleeve 66 to effect coaptation of the resulting valve leaflets.

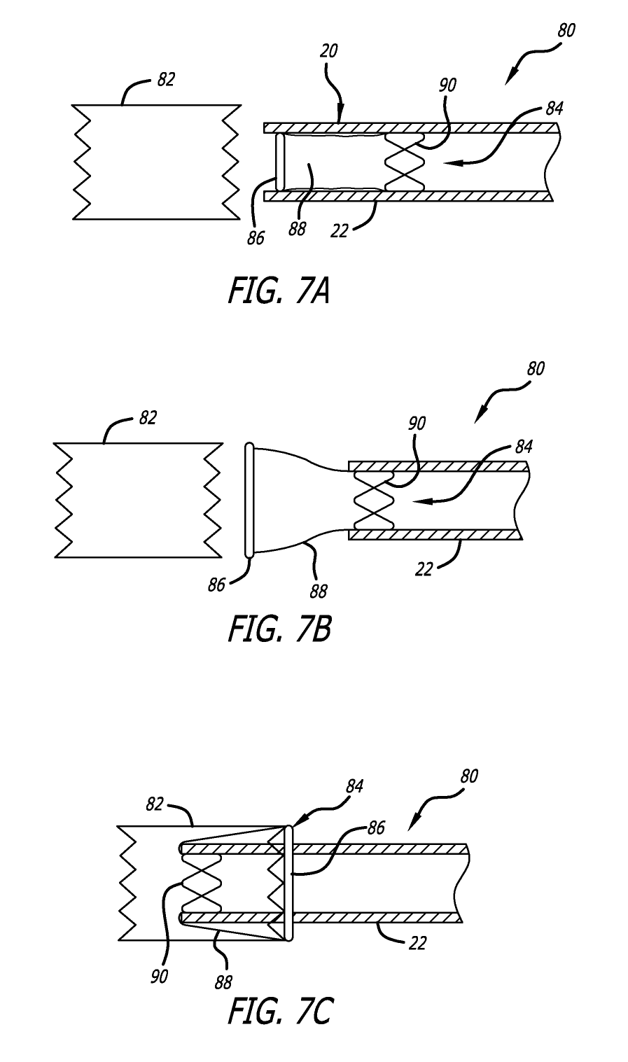

FIG. 6 shows a device 70 that avoids the use of a wireform. The device 70 of FIG. 6 generally includes a stent 72 and a valve assembly that includes an anchor ring 74 and a tissue sleeve 76 connecting the stent 72 and the anchor ring 74. Upon deployment using any of the above methods, the stent 72 is expanded, the ring 74 is advanced into the stent 72, inverting the tissue sleeve 76, and the ring 74 is expanded. Pinch points 78 are formed in the tissue sleeve 76. The pinch points 78 create the formation of valve leaflets once the device is inverted and subjected to blood flow.

All of the devices heretofore described have been directed to designs that allow the device to be delivered in an axially, displaced, unassembled form and inverted and located upon delivery to create a finished device. These devices are thus directed toward a goal of being able to compress the devices into a small catheter, such as a 14 French catheter, for delivery. Potentially, however, areas where two components connect, such as the connection between the tissue sleeve and the stent, will have slight overlap that may result in additional thickness. Thus, the remaining embodiments described herein are directed to devices having stents and valves that are not connected to each other while they are inside the delivery catheter.

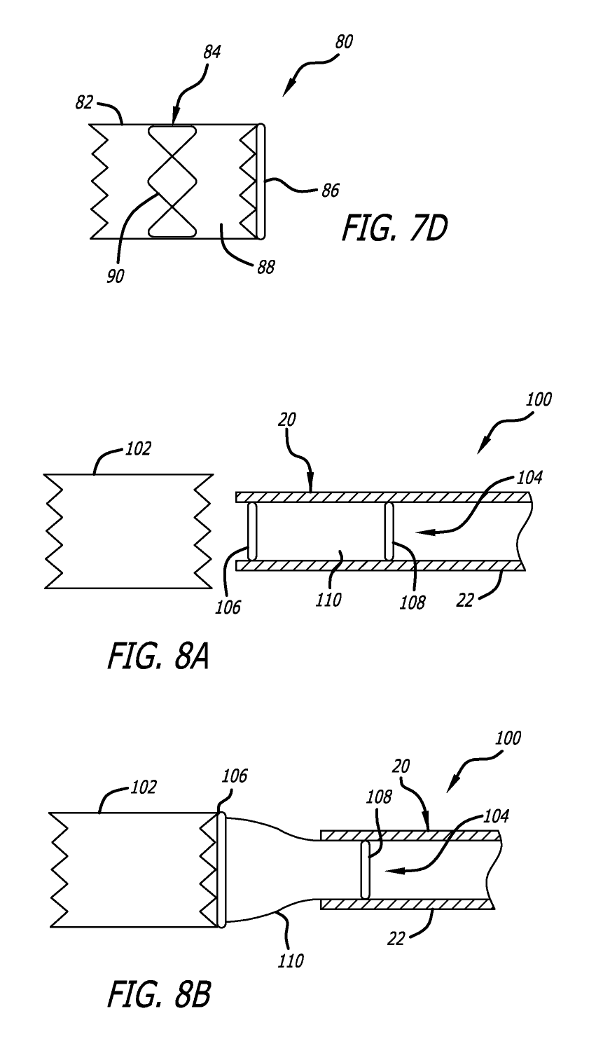

For example, referring to FIGS. 7A-D, there is shown a device 80 comprising two separate components: a stent 82 and a valve assembly 84. The valve assembly 84 includes a self-expanding anchor ring 86, a tissue sleeve 88 and a wireform 90. The anchor ring 86 anchors the valve assembly 84 in place until the wireform 90 is delivered. The anchor ring 86 also ensures proper reverse flow into the valve to effect coaptation of the resulting leaflets.

The delivery sequence for device 80 is as follows: As seen in FIG. 7A, a stent 82 has been placed at the native valve site. The delivery catheter 20 is advanced until the distal end of the catheter is near of the proximal end of the stent 82. The sheath 22 of the delivery catheter 20 is then retracted releasing the ring 86. (FIG. 7B) The ring expands just outside, or just inside of the stent 82. The delivery catheter 22 is advanced into the stent 82, causing the tissue sleeve 88 to invert. (FIG. 7C) The wireform 90 is then released from the sheath 22 and allowed to expand inside the stent 82 (FIG. 7D) and delivery is complete. The position of the wireform 90 relative to the tissue sleeve 88 constrains the tissue in such a way that the tissue sleeve is formed into valve leaflets. Conversely, the wireform 90 may have tissue leaflets already mounted to it and the tissue sleeve 88 is used solely to prevent perivalvular leak.

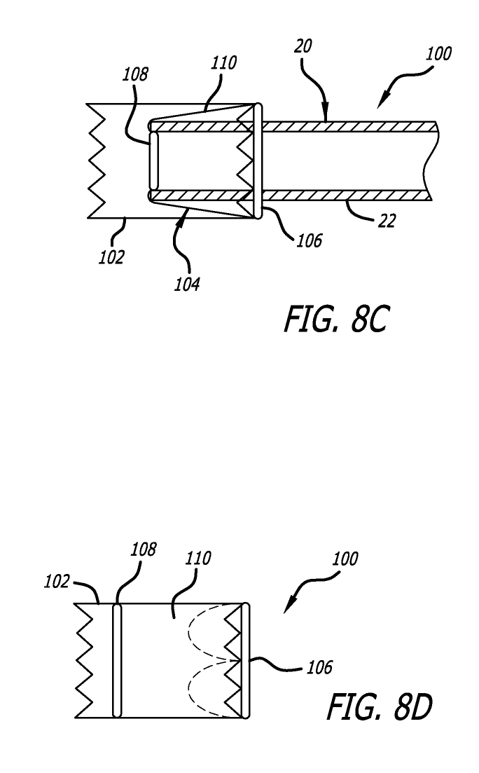

FIGS. 8A-D show an embodiment 100 that does not use a wireform. Rather device 100 comprises two separate components: a stent 102 and a valve assembly 104. The valve assembly 104 includes a first anchor ring 106, a second anchor ring 108, and a tissue sleeve 110 between the two anchor rings. The first anchor ring 106 anchors the valve assembly 104 in place to allow the tissue to be inverted.

The delivery sequence for device 100 is as follows: As seen in FIG. 8A, a stent 102 has been placed at the native valve site. The delivery catheter 20 is advanced until the distal end of the catheter is near of the proximal end of the stent 102. The sheath 22 of the delivery catheter 20 is then retracted releasing the ring 106. (FIG. 8B) The ring expands just outside, or just inside of the stent 102. The delivery catheter 22 is advanced into the stent 102, causing the tissue sleeve 110 to invert. (FIG. 8C) The second ring 108 is then released from the sheath 22 and allowed to expand inside the stent 102 (FIG. 8D) and delivery is complete. The tissue sleeve 110 may have attachment points at discrete multiple locations 112 around the circumference of the ring 106 in order to define the commissural points of the prosthetic tissue valve.

Though expanding the first ring 106 on the aortic (proximal) side of the stent 102 may be advantageous in order to establish initial alignment, one could avoid the inversion step by deploying the first anchor ring 106 on the ventricular, or distal side of the sent 102 and then further retracting the sheath 22 until the second ring 108 is released and allowed to expand near the aortic side of the stent 102.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A device for replacing a native valve comprising:
a stent;
a tissue sleeve; and,
an anchoring mechanism usable to secure said tissue sleeve within said stent;
wherein, in a configuration inside a delivery catheter, said anchoring mechanism is not located within said stent; and
wherein, in a deployed configuration, said tissue sleeve is located within said stent.

2. The device of claim 1 wherein in said deployed configuration, said tissue sleeve is inverted relative to said configuration inside said delivery catheter.

3. The device of claim 1 wherein said tissue sleeve is connected to said stent at a first end and connected to said anchoring mechanism at a second end, opposite said first end.

4. The device of claim 1 wherein said anchoring mechanism comprises a wireform.

5. The device of claim 1 wherein said anchoring mechanism comprises a ring.

6. The device of claim 1 wherein said anchoring mechanism comprises a ring and a wireform.

7. The device of claim 1 wherein said anchoring mechanism comprise a ring attached to a first end of said tissue sleeve and a second ring attached to an opposite side of said tissue sleeve.

8. The device of claim 1 wherein said tissue sleeve comprises valve leaflets.

9. The device of claim 1 wherein said tissue sleeve comprises pinch points that result in the formation of valve leaflets when implanted.

10. A method for replacing a native blood valve comprising:
expanding a stent within said native valve;
anchoring a tissue sleeve at a proximal end of said stent;
advancing said tissue sleeve into said stent, resulting in an inversion of said tissue sleeve;
allowing blood to flow through said tissue sleeve in a proximal direction while not allowing blood to flow through said tissue sleeve in a distal direction.

11. The method of claim 10 wherein expanding a stent within said native valve comprises expanding a stent within said native valve prior to introducing said tissue sleeve at a proximal end of said stent.

12. The method of claim 10 wherein anchoring a tissue sleeve at a proximal end of said stent occurs prior to the step of expanding a stent within said native valve.

13. The method of claim 10 wherein anchoring a tissue sleeve at a proximal end of said stent comprises attaching said tissue sleeve to said proximal end of said stent.

14. The method of claim 10 wherein anchoring a tissue sleeve at a proximal end of said stent comprises allowing a ring to expand at a proximal end of said stent, said ring attached to said tissue sleeve.

15. The method of claim 10 wherein allowing blood to flow through said tissue sleeve in a proximal direction while not allowing blood to flow through said tissue sleeve in a distal direction comprises providing valve leaflets attached to said tissue sleeve such that said valve leaflets are located on an outside surface of said tissue sleeve prior to said inversion and on an inside of said tissue sleeve after inversion.

16. The method of claim 10 wherein allowing blood to flow through said tissue sleeve in a proximal direction while not allowing blood to flow through said tissue sleeve in a distal direction comprises expanding a wireform within said tissue sleeve, said wireform creating valve leaflets in said tissue sleeve when expanded.

17. The method of claim 10 wherein allowing blood to flow through said tissue sleeve in a proximal direction while not allowing blood to flow through said tissue sleeve in a distal direction comprises providing pinch points in a proximal end of said tissue sleeve, after said tissue sleeve is inverted, said pinch point causing blood flow to form valve leaflets out of said tissue sleeve when blood is flowing in a distal direction.

18. The method of claim 10 further comprising the step of anchoring said tissue sleeve near a distal end of said stent after the step of advancing said tissue sleeve into said stent.

19. A valve assembly for implantation within a stent comprising:
a tissue sleeve;
at least one anchoring mechanism for securing said tissue sleeve within said stent;
wherein said tissue sleeve is connected to said at least one anchoring mechanism.

20. The valve assembly of claim 19 wherein said tissue sleeve is attached to a proximal end of said stent.

21. The valve assembly of claim 19 wherein said at least one anchoring mechanism comprises a wireform.

22. The valve assembly of claim 19 wherein said at least one anchoring mechanism comprises at least one expandable ring.

23. The valve assembly of claim 22 wherein said at least one expandable ring comprises a first expandable ring at one end of said tissue sleeve and a second expandable ring at another end of said tissue sleeve.

24. The valve assembly of claim 19 wherein said tissue sleeve comprises valve leaflets.

25. The valve assembly of claim 19 wherein said tissue sleeve forms valve leaflets when said at least one anchoring mechanism is expanded.

26. The valve assembly of claim 19 wherein said tissue sleeve comprises valve leaflets that become functional after said tissue sleeve is inverted.
